(19) United States
(12) Patent Application Publication
(10) Pub. No.: US 2013/0149765 A1
(43) Pub. Date: Jun. 13, 2013

(54) ANAEROBIC DIGESTER FOR THE PRODUCTION OF METHANE GAS FROM ORGANIC WASTE

(71) Applicant: Gary N. Austin, Stillwater, OK (US)

(72) Inventor: Gary N. Austin, Stillwater, OK (US)

(21) Appl. No.: 13/469,830

(22) Filed: May 11, 2012

Related U.S. Application Data
(60) Provisional application No. 61/485,107, filed on May 11, 2011.

(51) Int. Cl. C12M 1/107

(52) U.S. Cl. CPC C12M 21/04
USPC 435/167; 435/290.3

(57) ABSTRACT

An anaerobic digester to produce methane gas from animal manure. The anaerobic digester preferably includes a reactor vessel with a solar concentrator and an RF emitter. The reactor vessel may be loaded from the top with animal manure, sealed and evacuated during which the waste may be subject to focused light energy and/or subject to RF Energy from the RF emitter in order to facilitate anaerobic digestion, and then cleaned out with an auger turning out the resultant waste solids. An enzyme catalyst may also be employed to further facilitate anaerobic digestion. Methane gas produced as a result of anaerobic digestion is pumped from the reactor vessel and collected for further processing and/or burned for energy and/or heat.

### CROSS REFERENCE TO RELATED APPLICATION

This application claims the benefit of U.S. Provisional Application No. 61/485,107 filed May 11, 2011, herein incorporated by reference in its entirety for all purposes.

### FIELD OF THE INVENTION

The present invention relates generally to systems and methods for the anaerobic digestion of organic waste.

### BACKGROUND OF THE INVENTION

The process of anaerobic digestion of organic waste, such as manure produced by animals, to subsequently produce methane gas has been observed for many years. Indeed, it is understood that Native Americans of the North America Continent observed that a hotter camp-fire was achieved from supplementing a wood fire with buffalo chips, or simply burning the buffalo chips when wood was not available, because of the BTU released by the burning buffalo dung. Further, during World War II, Germany produced a significant amount of its farm fuel energy requirements from methane gas derived from animal manure and other organic waste.

Anaerobic digestion is a natural process in which bacteria break down organic matter in an oxygen-free environment to form biogas and digestate. A broad range of organic inputs can be used including manure, food waste, and sewage, with the composition being determined by the industry, agriculture, industrial, wastewater treatment, or others. Anaerobic digesters can be designed for either mesophilic or thermophilic operation—at 35° C. (95° F.) or 55° C. (131° F.), respectively. Temperatures are commonly regulated during the digestion process to keep the mesophilic or thermophilic bacteria alive. The resulting biogas is combustible and can be used for heating and electricity generation, or can be upgraded to renewable natural gas and used to power vehicles or supplement the natural gas supply. The resultant digested waste can be sold or used as fertilizer.

Anaerobic digestion, or decomposition, occurs in three stages. The first stage consists of hydrolysis and acidogenesis, where enzyme secreting bacteria convert polymers into monomers like glucose and amino acids and then these monomers are transformed into higher volatile fatty acids. The second stage is acetogenesis, in which bacteria called acetogens convert these fatty acids into hydrogen (H₂), CO₂, and acetic acid. The final stage is methanogenesis, where bacteria called methanogens use H₂, CO₂, and acetate to produce biogas, which is around 55-70 percent methane (CH4) and 30-45 percent CO₂.

It is estimated that animals that are grain fed in the stall can produce organic waste having a methane gas content which is upwards of 120 octane. Prior art waste digestion systems have principally included pits dug to fill with manure employing preferably thermophilic anaerobic digestion. The evolved methane gas was then collected off the top with hoses and/or tarps. Other attempts, including storage tanks that have been more effectively sealed against the loss of evolved methane have been shown to be too sophisticated and/or expensive for the average farmer/rancher to be able to afford and to use effectively.

A need, therefore, exists for an anaerobic digester which efficiently and economically controls the evolution of methane gas from organic waste such that the methane gas can be used for energy production and/or burned for heat.

### SUMMARY OF THE INVENTION

The present invention includes an anaerobic digester to produce methane gas from organic waste such as compost and animal manure. The anaerobic digester preferably includes a reactor vessel having at least one opening for input and discharge of the organic waste, an auger for turning the organic waste, a solar concentrator and an RF emitter. The reactor vessel may also include a heating tube which contains a liquid such as a mixture of water and antifreeze in a closed system. The heating tube may be in fluid communication with a reservoir and a solar collector used to heat the liquid which is then circulated through the heating tube. A pump may be used both to evacuate air from the reactor vessel and to pump out the gasses (including methane) evolved from the organic waste. The reactor vessel may be loaded from the top with organic waste such as compost or animal manure, sealed and evacuated during which the waste may be subject to focused light energy and/or subject to RF Energy from the RF emitter in order to heat up the organic waste to facilitate anaerobic digestion. Then once digestion of the organic waste is completed, the interior of the reactor vessel is evacuated with an auger turning out the resultant waste solids. An enzyme catalyst may also be added to the organic waste to further facilitate anaerobic digestion. Methane gas produced as a result of anaerobic digestion is pumped from the reactor vessel and collected, scrubbed for impurities, and stored for further processing. As a result, a supply of methane gas is provided which may be burned for energy and/or heat.

A method for the production of methane gas through anaerobic digestion of an inorganic waste feedstock includes the steps of: (a) obtaining a reactor vessel having an interior and an exterior; (b) the reactor vessel being capable of being sealed in order to contain an organic waste feedstock in a substantially airtight environment; (c) loading the reactor vessel with the organic waste feedstock; (d) evacuating the interior of the reactor vessel including the organic waste feedstock to provide a substantially airtight environment so as to facilitate anaerobic digestion of the organic waste contained therein; (e) receiving light energy from the sun onto the exterior of the vessel which may be painted in a light absorbing color, such as black, to heat the vessel and particularly the organic waste contained therein; (f) receiving light energy through a solar collector/concentrator into the interior of the vessel to heat the organic waste contained therein; (g) directing RF energy into the interior of the reactor vessel from an RF emitter so as to heat the organic waste contained therein; (h) evacuating the gasses evolved from the organic waste as a result of anaerobic digestion from the interior of the reactor vessel; and (i) discharging the digested organic waste once anaerobic digestion is complete.

### BRIEF DESCRIPTION OF THE DRAWINGS

FIG. 1 is a side view of the anaerobic digester of the present disclosure with the reactor vessel depicted partially cut away to show its interior and particularly the auger and heating tube therein.

FIG. 2 is a view taken along line 2-2 of FIG. 1.

FIG. 3 is an end view of the anaerobic digester of the present disclosure taken along line 3-3 of FIG. 1 depicted with the outlet door closed, top closed and reflective cover extended.

FIG. 4 is an end view of the anaerobic digester of the present disclosure of FIG. 3 depicted with the outlet door open, top open and the reflective cover retracted.

FIG. 5 is a partially exploded isometric view of the reactor vessel of the anaerobic digester of the present disclosure wherein one of the solar collector assemblies is depicted in an exploded view.

FIG. 6 is an isometric view of the anaerobic digester of the present disclosure depicted with the top lid of the reactor vessel in an open position.

FIG. 7 is a side view detail of the auger assembly of the anaerobic digester of the present disclosure.

FIG. 8 is a schematic diagram depicting the flow of the evolved gases produced by the anaerobic digester of the present disclosure.

FIG. 9 is a side view of the anaerobic digester of the present disclosure depicted partial cut away, to reveal the auger assembly, heater tubes, and solar concentrator assemblies.

FIG. 10 is a side view of an exemplary multi-stage plug flow anaerobic digester system wherein three separate anaerobic digesters of the present disclosure are connected in series.

FIG. 11A is a detailed of the sealing partition for the plug flow system of FIG. 10 in a closed position.

FIG. 11B is a detailed view of the sealing partition of the plug flow system of FIG. 10 depicting the partition in the open position in phantom.

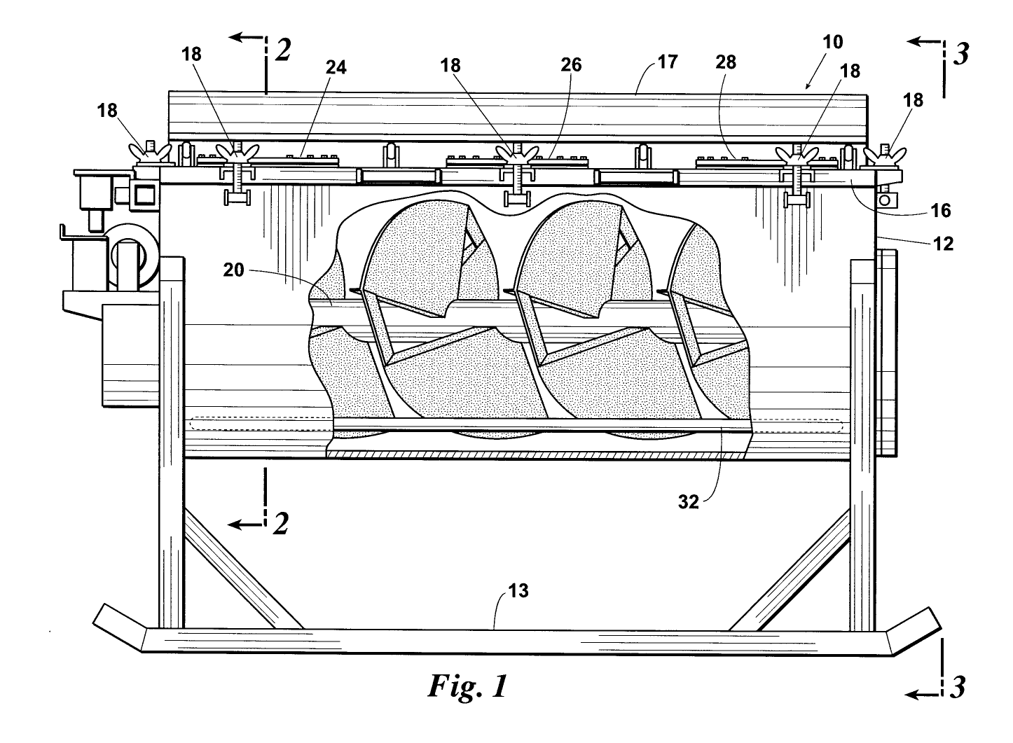

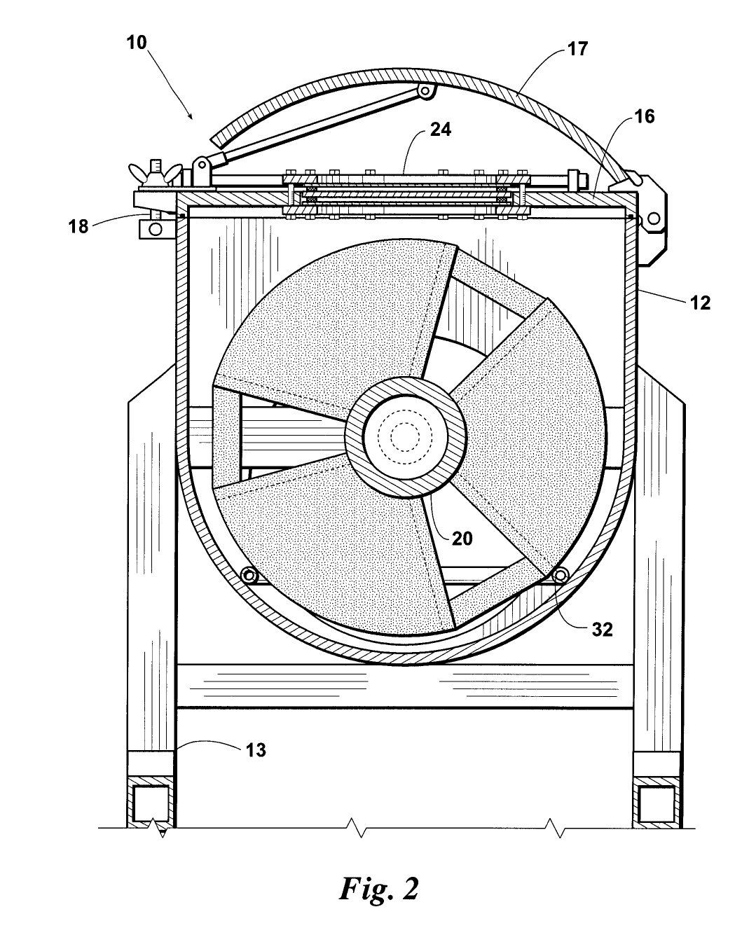

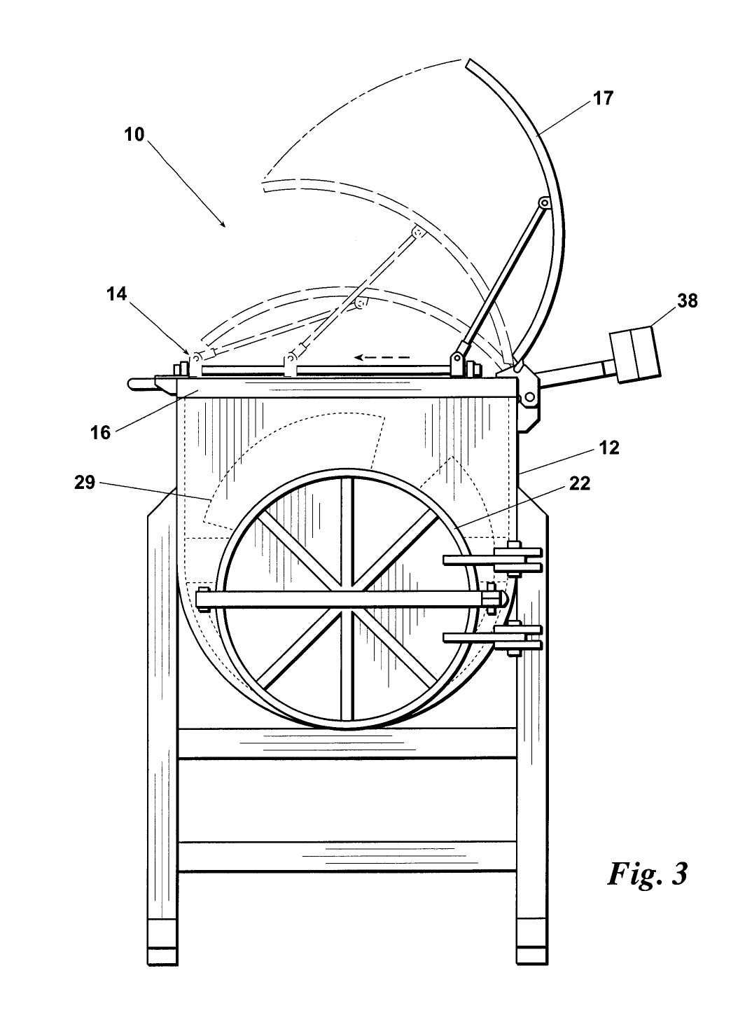

### DETAILED DESCRIPTION OF THE PREFERRED EMBODIMENTS

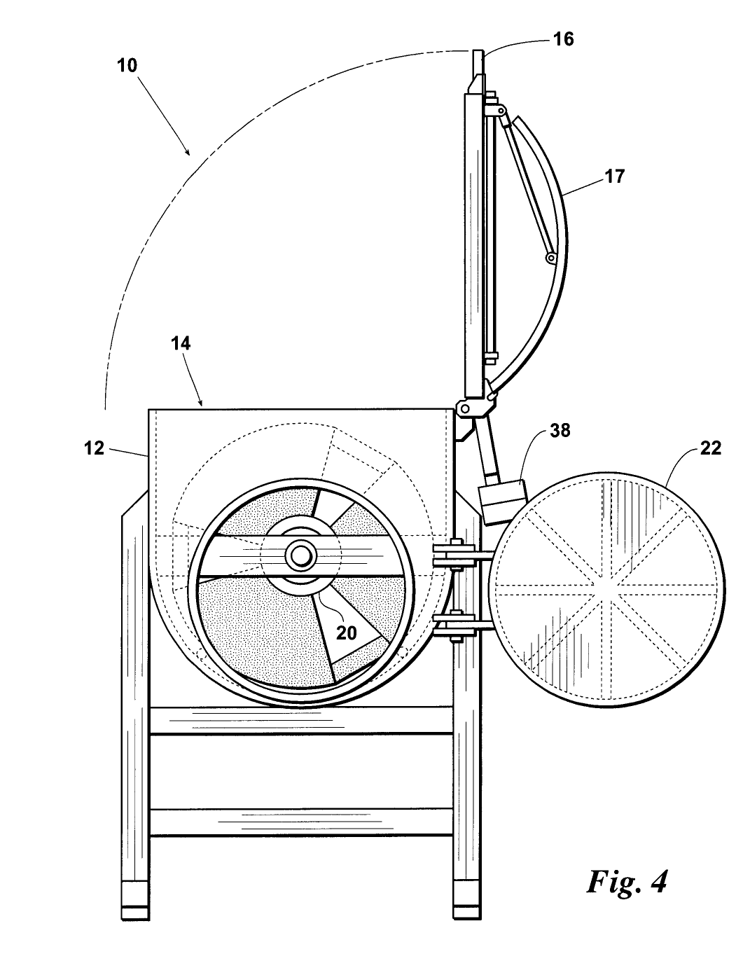

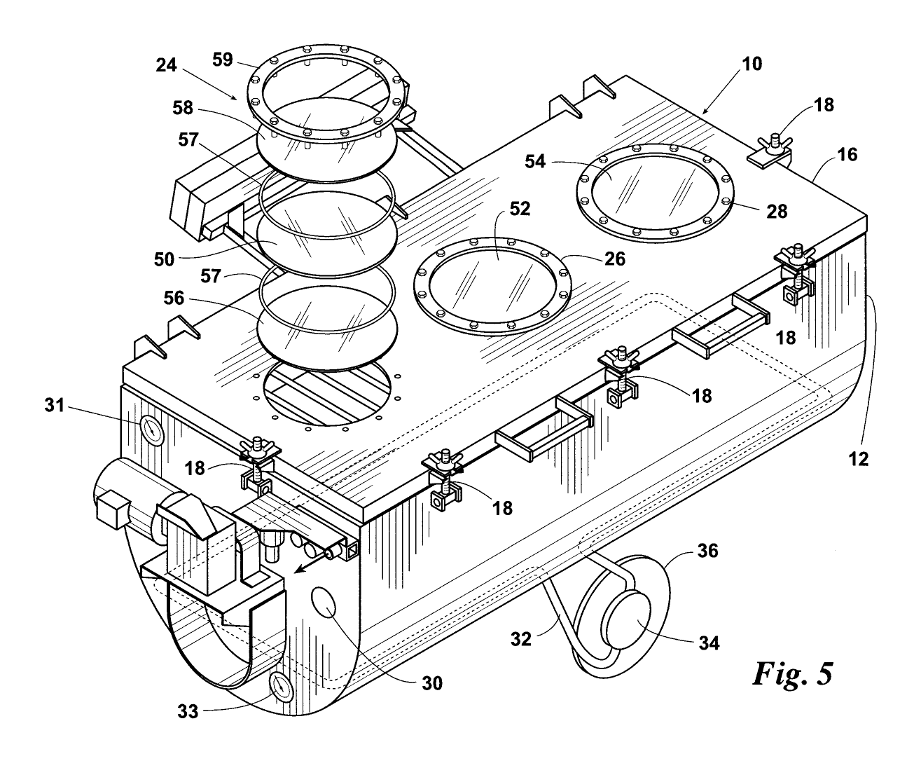

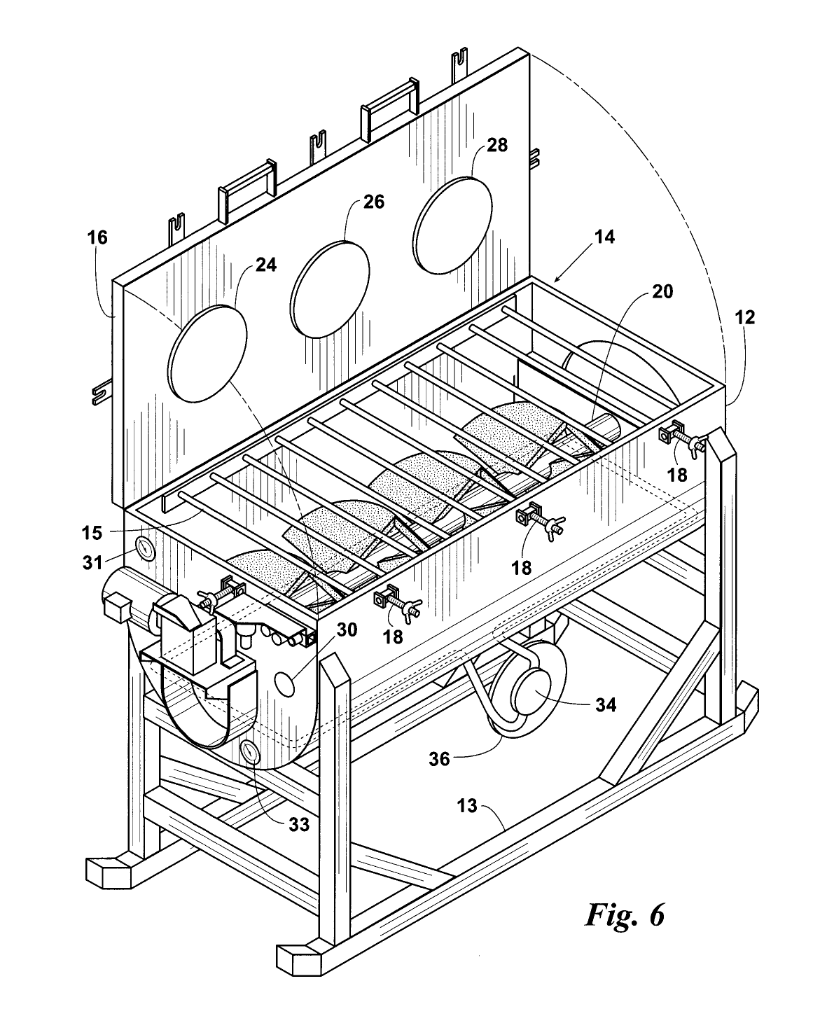

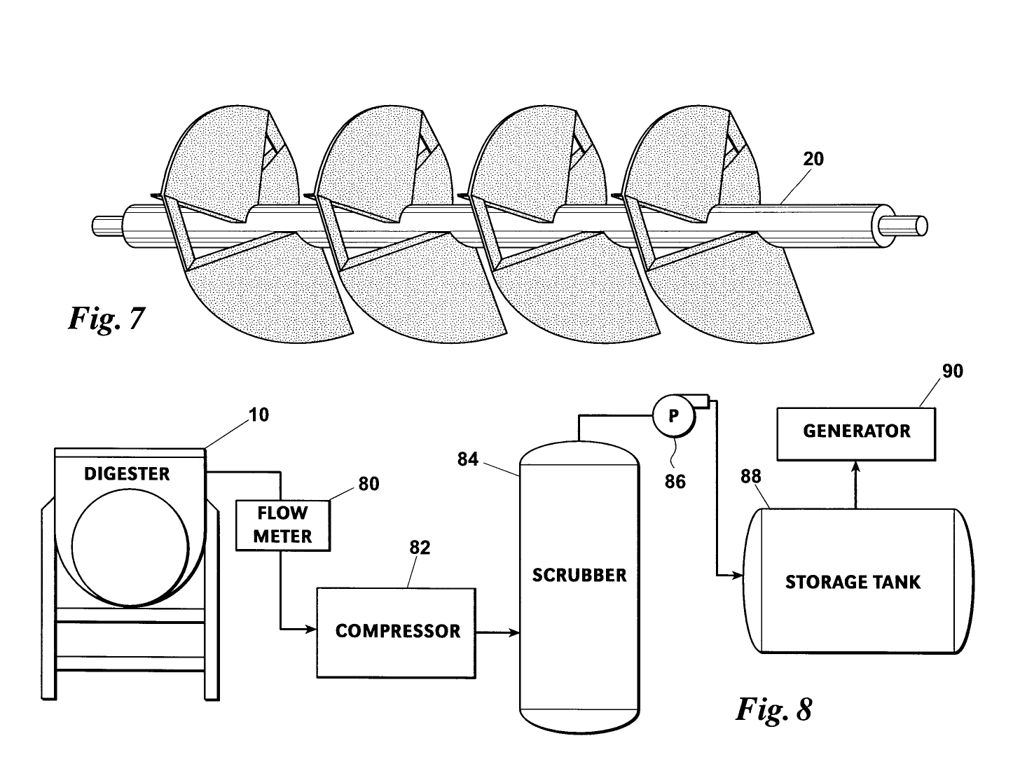

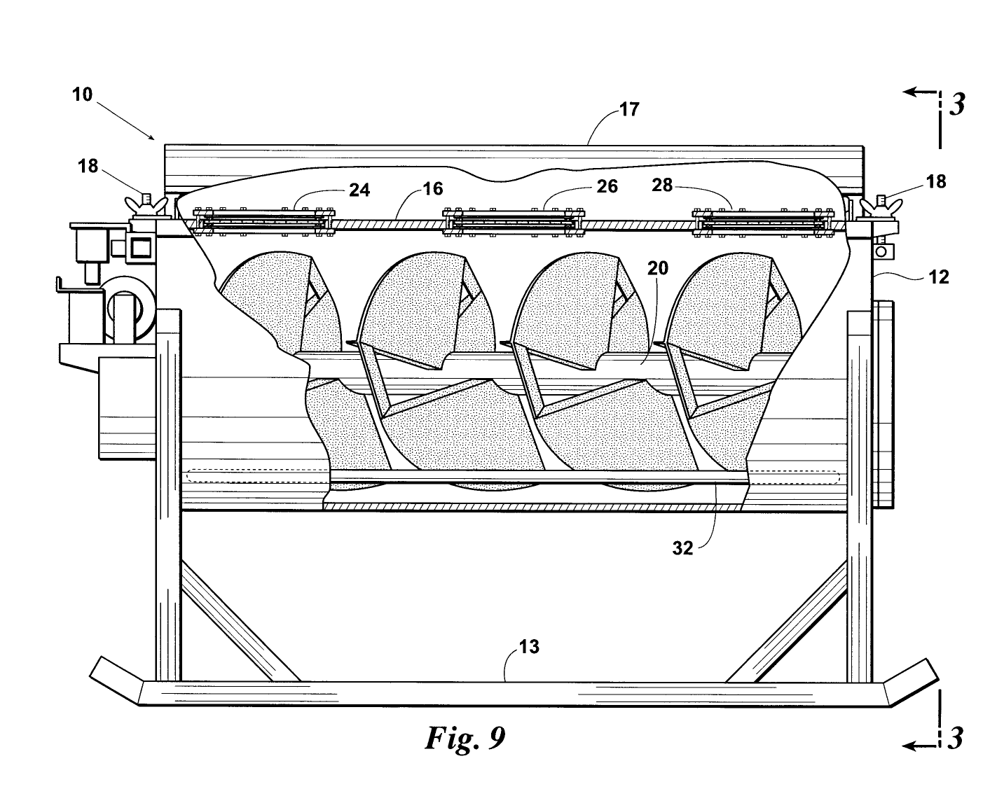

With reference to the Figures, wherein an anaerobic digester 10 used to produce methane gas from organic waste such as compost and animal manure is disclosed. Anaerobic digester 10 preferably includes a reactor vessel 12 having a top opening (14 of FIG. 4) for input of organic waste. A grate 15 is placed over top opening 14 under lid 16 but above auger 20. A retractable solar reflector 17 is mounted on lid 16 to collect and direct solar energy onto the exterior or reactor vessel 12. Reactor vessel 12 is secured by a lid 16 which may be sealed and secured by latches, collectively 18. A counterweight 38 facilitates opening and closing of lid 16. Reactor vessel 12 also includes an auger assembly 20 for turning the organic waste during digestion. An exit hatch 22 secures the end of reactor vessel 12 and provides a means for discharge of organic waste from the interior of reactor vessel 12. A suitable apparatus may be employed to secure exit hatch 22 such as bar 9 and latch 11. Reactor vessel 12 may be supported by any known structure, such as skid frame 13; however, other structures are contemplated and could even include wheels/tires for portability or other such means. Skid frame 13 can be sized to support reactor vessel 12 at any desired height or orientation

A plurality of solar concentrator assemblies 24, 26, and 28 are included on lid 16 for the collection, concentration, and direction of solar energy into the interior of reactor vessel 12. An RF emitter 30 may emit RF energy into the interior of reactor vessel 12. Since heat is added to the digestate, the process of the present disclosure is thermophilic and operates preferably in the 120-160° range.

Solar concentrator 24, 26, and 28 each include a Fresnel lens 50, 52, and 54 collect and concentrate solar energy into the interior of reactor vessel 12. The Fresnel lens 50 is preferably positioned (sandwiched) between transparent, protective sheets 56 or 58. Since reactor vessel 12 is designed to collect methane gas, it is preferable that transparent protective sheets 56 or 58 be constructed of a strong material such as Lexan® polycarbonate to protect Fresnel lens 50.

In a preferred arrangement, the Lexan® polycarbonate protective sheets 56 and 58 will have an abrasion resistant coating such as Nomar® or the like. Spacers 57 are preferably inserted between protector sheet 56 and lens 50, as well as between lens 50 and protective sheet 58. Solar concentration assembly 24 is then retained in lid 16 by way of collar 59 secured in a suitable fashion such as bolts. Solar concentrator assembly 24 is retained in lid 16 and lid 16 secured to reaction vessel 12 so as to be sealed so that anaerobic digestion can proceed within reaction vessel 12.

Reaction vessel 12 in the preferred arrangement is not a pressure vessel and is preferably designed to withstand pressures of up to approximately 10 psi. However, in a preferred arrangement, the pressure within reactor vessel 12 is maintained such that evolved gas pressure does not exceed a predetermined pressure of approximately 1.5 psi in the preferred embodiment. A pressure gauge 31 measures and indicates the pressure within reactor vessel 12. When pressure within reactor vessel 12 reaches the predetermined pressure such as approximately 1.5 psi, in a preferred arrangement, a pump is engaged to evacuate the evolved gas until the pressure within reactor vessel 12 drops below approximately 1.5 psi. Reactor vessel 12 also includes a pressure relief valve such that when the pressure within pressure vessel 12 reaches a second predetermined pressure, approximately 2 psi in a preferred arrangement, the pressure relief valve is automatically activated so as to vent evolved gas to the environment until the pressure within pressure vessel 12 drops below the predetermined pressure and the pressure relief valve closes. The same pump can be employed to evaluate reactor vessel 12 and pump out evolved gas.

Reactor vessel 12 may also include a heating tube 32 which contains a liquid such as a mixture of water and antifreeze in a closed system. Heating tube 32 may be in fluid communication with a reservoir 34 and a solar collector 36 used to heat the liquid which is then circulated through heating tube 32. A pump may be used both to evacuate air from reactor vessel 12 and to pump out the gasses (including methane) evolved from anaerobic digestion of the organic waste.

Reactor vessel 12 may be loaded from the top opening 14 with organic waste such as compost or animal manure, sealed and evacuated during which the waste may be subject to focused light energy from solar concentrator assembly 24, 26, and 28 and/or subject to RF Energy from RF emitter 30 in order to heat up the organic waste to facilitate anaerobic digestion. A temperature gauge 33 measures temperature within reactor vessel 12.

The area for the RF emitter unit 30 resonates, preferably, in the upper VHF spectrum with the use of an impedance matching device. Frequencies below 300 MHz and above 150 MHz are preferable. The power that could be used is preferably in the 100 to 500 watt range. At this power level, components are readily available commercially for RF emitter 30. A person of ordinary skill in the art would recognize that solid state devices represent the lowest cost approach. A suitable amplifier, MRFE6VP61K25H is available from Freescale, Inc., can produce up to 1 kW, and this amplifier with power supply is in the $2000 price range. For the amplifiers described, a matching circuit would be preferably in order to isolate the Digester from the output of the amplifier by matching the impedance of the two circuits. The impedance matcher would preferably be automated unless manual operation could be always used. The RF antenna could be a simple loop element, preferably in a basic embodiment. RF sensors would preferably be included to monitor heating as well as an RF shield for all open areas, including protection circuits. A motor 35 is in communication with auger 20 so as to rotate auger 20 to stir the organic waste during digestion. Stirring of the organic waste is accomplished on a predetermined schedule, such as every six hours such that constant stirring is not necessary. An explosion proof meter, ¾ hp, 175 rpm, 115/208/230 volt single phase is satisfactory, however, other motors can be employed.

Once digestion of the organic waste is completed, the interior of reactor vessel 12 is evacuated with auger 20 turning out the resultant waste solids through exit hatch 22. An enzyme catalyst may also be added to the organic waste to further facilitate anaerobic digestion with reference to FIG. 8, biogas, including methane gas, produced (evolved) as a result of anaerobic digestion is pumped from reactor vessel 12, compressed by a compressor 82, scrubbed for impurities in a scrubber 84, and stored in storage tank 86 for further processing such as used as fuel for a generator 90 to produce electricity. The CO₂ gas is separated from the methane gas and can also be sold or used for other purposes. As a result, a supply of methane gas is provided which may be used as fuel to provide electrical energy.

A method for the production of methane gas through anaerobic digestion of an inorganic waste feedstock includes the steps of: (a) obtaining a reactor vessel having an interior and an exterior; (b) the reactor vessel being capable of being sealed in order to contain an organic waste feedstock in a substantially airtight environment; (c) loading the reactor vessel with the organic waste feedstock; (d) evacuating the interior of the reactor vessel including the organic waste feedstock to provide a substantially airtight environment so as to facilitate anaerobic digestion of the organic waste contained therein; (e) receiving light energy from the sun onto the exterior of the vessel which may be painted in a light absorbing color, such as black, to heat the vessel and particularly the organic waste contained therein; (f) receiving light energy through a solar collector/concentrator into the interior of the vessel to heat the organic waste contained therein; (g) directing RF energy into the interior of the reactor vessel from an RF emitter so as to heat the organic waste contained therein; (h) evacuating the gasses evolved from the organic waste as a result of anaerobic digestion from the interior of the reactor vessel; and (i) discharging the digested organic waste once anaerobic digestion is complete.

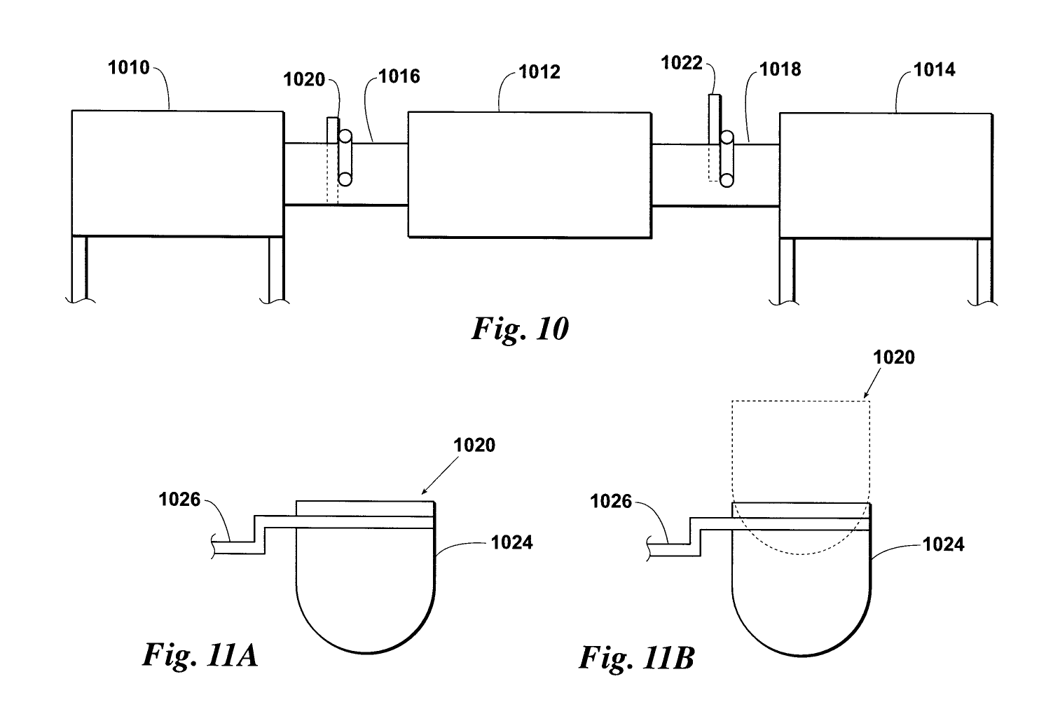

With reference to FIG. 10, a multi-stage plug flow anaerobic digester system shall next be described. In a multi-stage plug flow system, a plurality of reactor vessels 1010, 1012, and 1014 may be arranged in series so as to be spaced from and in communication via tunnels 1016 and 1018. As shown in greater detail in FIGS. 11A and 11B, tunnels 1016 and 1018 may be divided by a sealing partition 1020 and 1022. With reference to FIGS. 11A and 11B, exemplary sealing partition 1020 is depicted in its design therefor to divide, seal, and isolate the respective reactor vessels, 1010, 1012, and 1014 from one another. Exemplary sealing partition 1020 includes a sealing plate 1024 and a sealing mechanism such as a hand crank 1026. As such, sealing partition 1024 may be raised and lowered by hand crank 1026 in tube 1016 (FIG. 10) so as to divide tube 1016 and isolate reactor vessel 1010 from reactor vessel 1012. Likewise, sealing partition 1022 may be raised or lowered within tube 1018 so as to divide tube 1018 thus seal reactor vessel 1012 from reactor vessel 1014.

In operation, organic waste is added to reactor vessel 1010, which is then sealed and evacuated. Sealing partition 1020 is closed and anaerobic digestion of the organic waste within reactor vessel 1010 is allowed to commence. After a predetermined time, but before full anaerobic digestion of the organic waste within reactor vessel 1010 occurs, the organic waste within reactor vessel 1010 is discharged via an auger within reactor vessel 1010 into tube 1016. Sealing partition 1020 is raised and the organic waste may be pushed into tube 1016 and through tube 1016 into reactor vessel 1012. Sealing partition 1022 between reactor vessel 1012 and reactor vessel 1014 is closed. At this time, anaerobic digestion of the waste transferred from reactor vessel 1010 into reactor vessel 1012 continues in reactor vessel 1012. Sealing partition 1020 is once again closed and new undigested organic waste feedstock is supplied to reactor vessel 1010. Once again, reactor vessel 1010 is sealed and evacuated and anaerobic digestion is allowed to take place both within reactor vessel 1010 and reactor vessel 1012.

Prior to the complete digestion of organic waste in either reactor vessel 1010 or reactor vessel 1012 proceeds to completion, both sealing partitions 1020 and 1022 are raised in the organic waste contained within reactor vessel 1010 and reactor vessel 1012 are evacuated. The organic waste contained within reactor vessel 1012 is conveyed through tunnel 1018 to reactor vessel 1014 from reactor vessel 1012 while the organic waste contained within reactor vessel 1010 is conveyed via tunnel 1016 into reactor vessel 1012. At this time, sealing partitions 1020 and 1022 are again closed and sealed and anaerobic digestion of the organic waste within reactor vessel 1012 and 1014 is allowed to continue while new undigested organic waste feedstock is added to reactor vessel 1010. Reactor vessel 1010 is sealed and evacuated and anaerobic digestion is allowed to commence.

At the point in time when the organic waste contained within reactor vessel 1014 proceeds to complete digestion, the organic waste contained within reactor vessel 1014 is evacuated from reactor vessel 1014 and is prepared for further use or sale. The partition 1022 between reactor vessel 1012 and reactor vessel 1014 is once again opened and the organic waste contained within reactor vessel 1012 is conveyed into reactor vessel 1014. Both reactor vessel 1012 and reactor vessel 1014 are then sealed and evacuated and anaerobic digestion within reactor vessel 1014 is allowed to continue. Partition 1020 is then opened and the contents of reactor vessel 1010 is then conveyed into reactor vessel 1012, partition 1020 closed and the anterior of reactor vessel 1012 evacuated. Once again new organic waste feedstock is supplied to reactor vessel 1010 which is then sealed and evacuated so that anaerobic digestion commences therein.

Exemplary economic benefits derived from anaerobic digester 10 of the present disclosure shall next be described. First, the digested feedstock has value as a fertilizer compost for agriculture.

An exemplary anaerobic digester of the present disclosure including a 500 gallon reactor vessel has a volume of approximately 53 ft.³ of which 40 ft.³ is containable for digesting the animal manure feedstock to a fertilizer compost, and 13 ft.³ is for gas evolution. Once anaerobic digestion begins there is a reduction in waste solid volume.

Assuming the wholesale price of the fertilized compost to be $2.00 per bag at one cubic foot each, 40 ft.3×$2.00=$80.00 per load digested, times three loads per month=$240.00×12 months=$2,880.00 per year.

In addition, and in particular, the evolved methane gas has economic value for energy production and/or heating. Methane fuel gas (CH₄) has a heating value of 1,048 Btu/ft.³ however only 65% of the gas produced from the animal manure is expected to be CH₄ with the other gases anticipated to be 30% CO₂ (carbon dioxide) and 5% H₂S (hydrogen sulfide).

A one thousand pound horse produces approximately 50 pounds of manure/day. Gas yield is 15 ft.³/lb. Therefore 50 lb.×15 ft.³=750 ft.³ of gas.
x 0.65
487.5 ft.³ of CH₄

Horse manure weighs 63 lb./ft.³

For the purposes of example herein, Reactor vessel 12, the digester tank holds 40 ft.³×63 lb.=2520 lb. of horse manure. This value is then divided by 50 lb.=50.4×487.5 ft.³=24570 ft.³ of CH₄.
X 3 loads/mo.
73710 ft.³/mo.×1,048 Btu/ft.³=77,248,080 BTU's.

This monthly production is then multiplied by 12 months to equal 926,976,960 Btu's/year. This result, rounded off equals 927 million BTU's.

As an example, assume natural gas is trading at $4.00/million BTU. Thus multiply 927 million×$4.00=$3,708.00/year. Adding the described values for the digested compost and the methane:

1) $2,880.00
2) +3,708.00
Result in $6,588.00 per year from a 500 gallon digester tank.

Increasing product yields by ⅓ using all of the same above variables for a 750 gallon digester tank will yield:

1) $3,840.00
2) +$4,944.00
$8,784.00

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are encompassed within the spirit of this invention as defined by the appended claims.

What is claimed is:

1. A reactor for the production of methane gas through anaerobic digestion, said reactor comprising:
a vessel capable of receiving, containing and discharging organic waste;
said vessel capable of containing said organic waste in a substantially airtight environment;
said vessel including at least one solar concentrator to focus light energy into said organic waste;
a pump in communication with said vessel for evacuating the vessel to facilitate anaerobic digestion.

2. The reactor of claim 1 wherein said solar concentrator includes at least one Fresnel lens.

3. The reactor of claim 2 wherein said vessel includes a lid and said at least one Fresnel lens is secured in said lid.

4. The reactor of claim 3 wherein said at least one Fresnel lens is positioned between transparent protective sheets.

5. The reactor of claim 4 wherein said transparent protective sheets are constructed of polycarbonate.

6. The reactor of claim 1 wherein said vessel includes at least one feed through RF antenna capable of emitting RF energy into said organic waste.

7. A reactor for the production of methane gas through anaerobic digestion, said reactor comprising:
a vessel having at least one opening to facilitate loading and unloading of said vessel with organic waste;
said vessel including at least one solar concentrator capable of directing light energy into said organic waste;
said vessel including at least one feed through antenna capable of emitting RF energy into said organic waste;
said vessel being capable of containing said organic waste in a substantially airtight environment.

8. The reactor of claim 7 wherein said solar concentrator includes at least one Fresnel lens.

9. The reactor of claim 8 wherein said vessel includes a lid and said at least one Fresnel lens is secured in said lid.

10. The reactor of claim 9 wherein said at least one Fresnel lens is positioned between transparent protective sheets.

11. The reactor of claim 10 wherein said transparent protective sheets are constructed of polycarbonate.

12. A method for the production of methane gas through anaerobic digestion of an organic waste feedstock comprising the steps of:
obtaining a reactor vessel having an interior and an exterior;
said vessel being capable of containing organic waste feedstock in a substantially airtight environment;
loading said reactor vessel with an organic waste feedstock;
evacuating said interior of said reactor vessel to provide said substantially airtight environment in order to facilitate anaerobic digestion of said organic waste;
at least intermittently receiving light energy onto said exterior of said reactor vessel to heat said organic waste;
at least intermittently directing light energy into the interior of said reactor vessel to heat said organic waste;
at least intermittently directing RF energy into the interior of said reactor vessel to heat said organic waste;
at least intermittently evacuating the gasses from the interior of said vessel which are evolved as a result of anaerobic digestion of said organic waste;
discharging digested organic waste from the interior of said vessel.
